(19) United States
(12) Patent Application Publication
(10) Pub. No.: US 2008/0311577 A1
(43) Pub. Date: Dec. 18, 2008

(54) Method for the Identification of Sulfo-Oxidizing Bacteria and for the Monitoring of Elemental Sulfur in the Environment

(75) Inventors: Antonella Crisari, Rome (IT); Francesca De Ferra, Lodi (IT)

Correspondence Address: OBLON, SPIVAK, MCCLELLAND MAIER & NEUSTADT, P.C., 1940 DUKE STREET, ALEXANDRIA, VA, 22314, US

(73) Assignee: ENI S.p.A., Rome (IT)

(21) Appl. No.: 11/995,042

(22) PCT Filed: Jul. 7, 2006
(86) PCT No.: PCT/EP06/06699

§ 371 Date: Mar. 10, 2008

(30) Foreign Application Priority Data
Jul. 8, 2005 (IT) MI2005A001294

(51) Int. Cl. C12Q 1/68; C07H 21/00

(52) U.S. Cl. 435/6; 536/24.3

(57) ABSTRACT

A method is described for the identification of sulfooxidizing bacteria comprising the extraction of the DNA from environmental samples and the subsequent identification of at least one fragment of the Thio 16S gene or SoxB gene present in these bacteria. The method can be used for determining the level of elemental sulfur in samples of soil.

The present invention relates to a method for the identification of sulfo-oxidizing bacteria in environmental samples by the identification of specific genes present in these bacteria.

Sulfo-oxidizing bacteria are known for being microorganisms which are greatly responsible for the oxidation, in natural environments, of elemental sulfur up until sulfuric acid (C. Parker and J. Prisk. The oxidation of inorganic compounds of sulfur by various sulfur bacteria J. Gen. Microbiol. 8: 344-346 (1953); J. Waksman and B. Joffe. Microorganism concerned in the oxidation of sulfur in the soil J. Bateriology 7, 239-256 (1952).

In this way, sulfur and sulfur-based compounds are solubilized in the form of sulfates and become available for assimilation on the part of other microorganisms and plants, thus contributing to the fertilization of the soil.

The biological oxidation of sulfur, however, can be harmful to the environment if the quantity of acid produced by the oxidation of sulfur exceeds the capacity of the soil for neutralizing this acidity.

This problem generally arises in places in which sulfur is recovered by the desulfuration of fossil fuels and left in deposit in large quantities.

As a result of biological oxidation on the part of sulfo-oxidizing bacteria, extremely acidic percolates are in fact formed in sulfur deposits, which derive from contact between rainwater and the surface of the sulfur.

Analogous phenomena also occur in the areas surrounding deposits where the sulfur can be easily dispersed by the action of the wind; all of this creates serious damage to the environment as well as a risk for people's health.

As sulfo-oxidizing bacteria are mainly responsible for acidification processes associated with the presence of sulfur, the methods for their identification and control are a valid means for following these phenomena and for handling the environmental risk due to the dispersion of sulfur.

A classical method for the identification of sulfo-oxidizing bacteria which comprise various species among which Thiobacilli, is based on the MPN (Most Probable Number) technique which consists in evaluating the most probable number of bacteria present in the soil through successive dilutions of a sample of a selective medium for Thiobacilli, in the presence of sulfur and a pH indicator which reveals that the acidification has taken place.

This method, however, requires lengthy operating times as, when the bacterial concentration is low, the incubation times can even reach four weeks (C. Knickerbocker. The role of blebbing in overcoming the hydrophobic barrier during bioxidation of elemental sulfphur by Thiobacillus Thiooxidans Chem. Geology 169:425-433 (2000)) and it is not very accurate due to the fact that, as widely described in literature, only as small fraction of the active microbial species in natural ecosystems can be easily cultivated in a laboratory.

Various methods are described in literature for the identification of sulfo-oxidizing bacteria which are based on genetic techniques such as, for example, the use of genes for resistance to mercury or resistance to arsenic as marker genes for Thiobacilli (M. Barrow. Production and regeneration of Thiobacillus ferroxidans spheroplasts. Applied and Environmental Microbiology 50:721-723 (1985)).

The use of genes for resistance to mercury (T. Barkay, D. Fouts. Preparation of a DNA gene probe for detection of mercury resistance genes in Gram-negative bacterial communities. Applied and Environmental Microbiology. 49:686-692 (1985)) is based on a DNA probe capable of identifying them (mer-probe). This gene however cannot be considered as being specific for Thiobacilli and it only allows gram-negative bacteria (which have mer-probe) to be distinguished from gram-positive bacteria (which do not have this).

The method which uses arsenic resistance as marker is based on the transformation of Thiobacillus with a plasmid and the subsequent detection of the plasmid itself. It is therefore a laborious method which can only be used in experimentation situations and not for environmental monitoring.

The use of gene probes detected with FISH (Fluorescent In Situ Hybridization) is also described, which recognize specific sequences of 16SRNA for Thiobacillus thiooxidans and a second sequence, (Thio 820), based on 16SRNA for identifying Thiobacillus thiooxidans and ferrooxidans (M. Hernandez, E. Marchand and J. Peccia. In situ assessment of active Thiobacillus species in corroding concrete sewers using fluorescent RNA probes International Biodeterioration and biodegradation 49: 271-276 (2002)). These identification methods however are only specific for these two species of Thiobacillus, whereas there are numerous known sulfo-oxidizing species and with different 16S sequences. In addition to this first and important consideration, it is also unthinkable to imagine bacterial monitoring campaigns based on the FISH technique, which is much more complicated and laborious for use than the techniques based on gene amplification provided by PCR, above all on a soil matrix.

The biological system of use of elemental sulfur on the part of microorganisms of the soil has not yet been completely clarified either on a genetic level or on a microbial ecology level.

For the setting up of a new sulfur monitoring method in the soil, it was therefore necessary to experimentally verify whether the gene dosage of a system which could be considered as being involved in oxidative reactions of sulfurated compounds (the gene system responsible for the expression of Sox genes) could be correlated to the presence and quantity of elemental sulfur in the soil.

A method has now been found, based on the recognition of specific genes such as Thio16S or SoxB, which enables the identification and quantification of sulfo-oxidizing bacteria with a high sensitivity, specificity and rapidity.

In particular, the method of the invention envisages that the genes Thio16S and SoxB be identified by means of the amplification of one of their fragments in the presence of specific primers.

It has also been found that the quantitative level of these genes can be correlated to the presence and quantity of elemental sulfur in the soil.

An object of the present invention therefore relates to a method for the identification of sulfo-oxidizing bacteria comprising the extraction of the DNA from environmental samples and the subsequent identification of at least one fragment of the Thio16S ribosomal gene or SoxB gene present in sulfo-oxidizing bacteria.

A further object of the present invention relates to a method for determining the dispersion of sulfur in the environment comprising the identification of sulfo-oxidizing bacteria as described above and their quantitative determination.

The identification of the fragment of the Thio16S gene is effected by means of gene amplification in the presence of a pair of oligonucleotides complementary to the Thio16S gene, selected from the following pairs of sequences:

| | |
|---|---|
| T 9 | GAA AAG GYG GGT YCT AAT A |
| T19 | CAT CTC TGC AAR RTT CCG G |
| T 9 | GAA AAG GYG GGT YCT AAT A |
| T 21 | CCT GTG TTC CGA TTC CCC GA |

whereas the identification of the SoxB gene is effected by means of gene amplification in the presence of pairs of oligonucleotides complementary to the SoxB gene in which the primer forward is selected from the following sequences:

| | |
|---|---|
| 360f | GAT CCT GTC GGG NCA YAC SCA YGA |
| 445f | TAT CGG CGS GGC AAY TTC AA |

and the primer reverse is selected from the following sequences:

| | |
|---|---|
| 445r | GTT GAA GTT GCC SCG SCG RTA |
| 531r | GTC GCC GCC TTG YTG RTA RTA |
| 529r | GCC CTG CTG GTA RTA SGG RTC |

The gene amplification of the SoxB gene is preferably effected in the presence of pairs of oligonucleotides selected from the following pairs of sequences:

| | |
|---|---|
| 360f | GAT CCT GTC GGG NCA YAC SCA YGA |
| 445r | GTT GAA GTT GCC SCG SCG RTA |
| 360f | GAT CCT GTC GGG NCA YAC SCA YGA |
| 531r | GTC GCC GCC TTG YTG RTA RTA |
| 445f | TAT CGG CGS GGC AAY TTC AA |
| 529r | GCC CTG CTG GTA RTA SGG RTC |
| 445f | TAT CGG CGS GGC AAY TTC AA |
| 531r | GTC GCC GCC TTG YTG RTA RTA |

The gene amplification techniques (Polymerase Chain Reaction—PCR) have the advantage, with respect to the more widely used MPN bacterial culture techniques (long and often unreliable), of being able to also identifying not easily cultivable species which often represent an important fraction of the bacteria present in the ecosystem.

In order to be used generically, PCR-based techniques must succeed in only identifying target genes and, preferably, all the species containing them.

The method of the invention has revealed a greater sensitivity, specificity and rapidity with respect to the methods described in the known art (MPN) and can also be conveniently used for following the dispersion of sulfur in the environment.

A further object of the present invention relates to oligonucleotides having a sequence selected from those indicated above.

These oligonucleotides can be used not only as primers for gene amplification, but also as gene probes for the identification of the Thio16S gene and the SoxB gene of sulfo-oxidizing bacteria.

In this case, when using the techniques of the known art, the oligonucleotides of the invention are subjected to marking so that they can be easily detected and subsequently subjected to hybridization with the genomic DNA to be analyzed, or that obtained through gene amplification, (such as in the FISH technique for example) which allows specific sequences to be identified by fluorescence in samples containing cells or microorganisms as described for example in “In Situ Hybridization. A practical Approach” Edited by D-G. Wilkinson IRL Press, Oxford University Press, 1994.

The marking can be effected with different techniques such as, for example, fluorescence, radioactivity, chemiluminescence or enzymatic marking.

The use of specific probes for the SoxB gene is preferable as they not only identify sulfo-oxidizing bacteria of the Thiobacillus kind, but also other microbial species potentially involved in the use of sulfur in environmental matrixes.

It is estimated, on the other hand, that Thiobacillus can form the predominant species in the soil in recycling reactions of elemental sulfur, and therefore the use of Thio16S probes should be capable of providing similar responses.

The combined use of two types of probes can provide, in selected samples, a control of the efficacy of the method.

The identification of the oligonucleotides of the invention has led to the use of various sequences available in data banks of the Thio16S gene and the SoxB gene.

The sequences were aligned with the use of Clustal X sequence analysis software (Thomson J D et al., Nucleic Acids Research, 24:4876-4882 (1994)), in order to define the regions preserved and identify, in homologous areas, the specific nucleotide sequences to be used as primers for selectively amplifying the Thio16S gene and the SoxB gene.

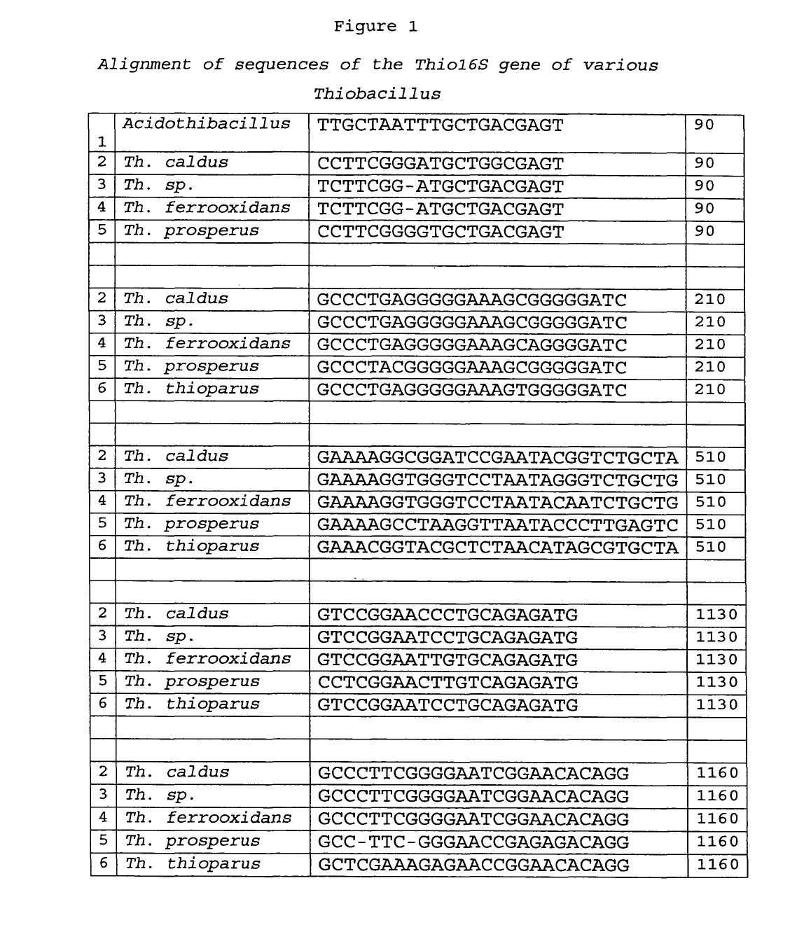

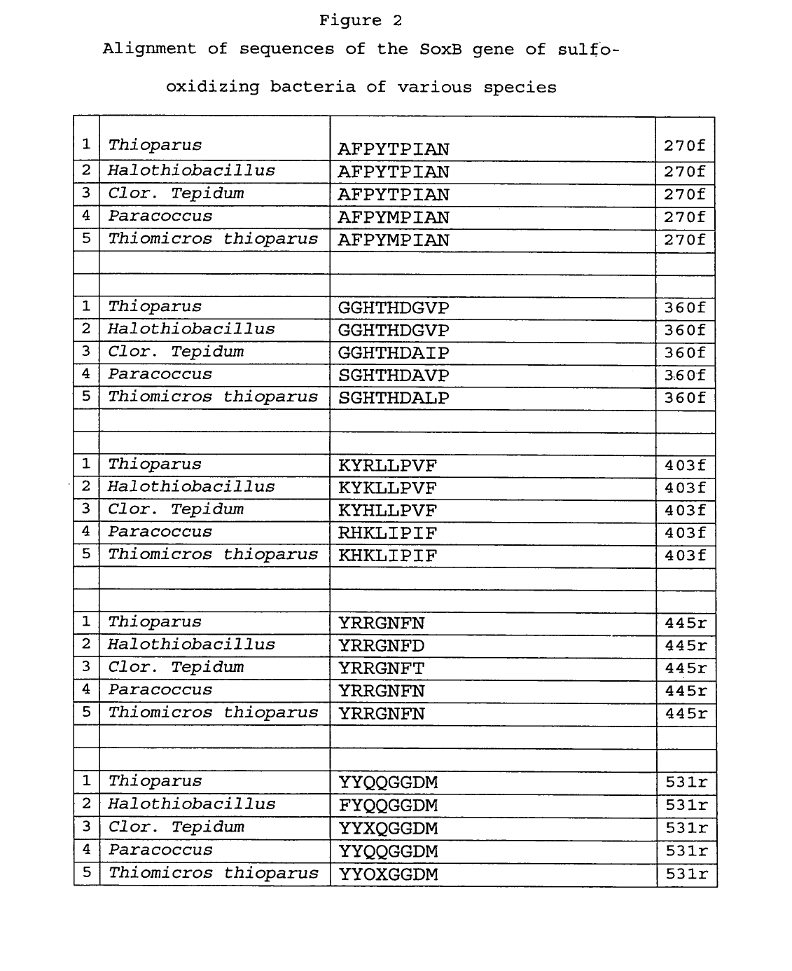

FIG. 1 indicates the alignment of the nucleotide sequences for the Thio16S gene in various Thiobacilli and FIG. 2 the alignment of the amino acid sequences for the SoxB gene in different sulfo-oxidizing bacteria.

On examining the homologous areas, different regions of the Thio16S gene and SoxB gene were identified with a preserved sequence from which the primers indicated respectively in Table 1 and Table 2 were synthesized.

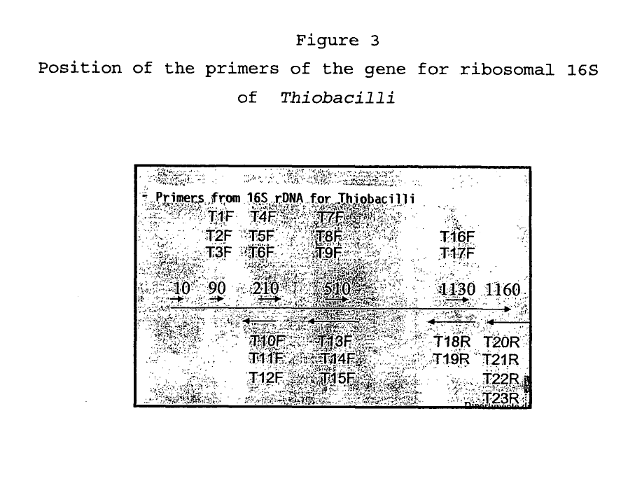

FIG. 3 shows the position of all the primers of Table 3 in the gene for the ribosomal 16S of Thiobacilli.

The detection method of sulfo-oxidizing bacteria of the present invention comprises, in particular, the following passages:

extraction of the DNA from environmental samples;

amplification of the DNA using the pairs of primers described above with PCR or with qPCR (quantitative PCR);

analysis by means of gel-electrophoresis of the results of the PCR or by means of immunofluorescence of the products obtained with qPCR.

The sample to be analyzed can consist of water or soil coming from environmental samplings, or from bacterial cultures.

The extraction of the genomic DNA from the samples to be analyzed can be effected with various techniques:

if cultures of Thiobacillus are used, the DNA is extracted with the CTAB/NaCl method (J. Wiley. Preparation of genomic DNA from bacteria Molecular Biology Unit 2.4. (1994)) and resuspended in a TE buffer (Tris Edta).

when starting from environmental samples (soil, water), the preferred method uses the Fast DNA Kit (BIO 101) commercialized by Qbiogene or similar methods and the DNA is resuspended in a DES buffer (DNA Elution Solution), whose principle is based on the adsorption of the DNA and subsequent elution from a resin.

Analysis with the primers object of the invention allows the detection times of sulfo-oxidizing bacteria to be considerably reduced, allowing their detection and quantification within a few hours, whereas the methods commonly used (MPN) which are based on the effective bacterial cultivability, require much longer times (up to 4 weeks).

The method used is based on the amplification of the DNA due to a DNA polymerase in the presence of a pair of specific primers (J. Sambrook, E. F. Fritsch and T. Maniatis. A laboratory manual. CSH Laboratory Press (1989)) and is commonly used for the detection of target sequences of DNA in various types of samples, for diagnostic purposes in medicine or in environmental analyses.

Temperature conditions, reaction times and additional agents have been set up, which are necessary for allowing the fragment of the Thio16S gene and SoxB gene to hybridize with the primers in a specific mode.

The conditions preferably adopted for the annealing temperature in the phase preceding the polymerization in the PCR reaction vary from −5 to −1° C. with respect to the lowest melting point Tm among those of the pair of probes used.

The preferred polymerization time depends on the length of the desired product, estimating about 1 kilobase of DNA polymerized per minute of reaction.

Conditions which allow “specific amplification” refer to conditions which prevent the amplification of different sequences from those of the Thio16S gene and SoxB gene.

The annealing during the amplification reaction is carried out at temperatures compatible with the sequence of the primers, in this specific case at 57° C. for Thio16S and at 55° C. for SoxB.

The buffers and enzymes used are solutions compatible with the characteristics of the DNA polymerase used, such as, for example, Taq polymerase, ampliTaq Gold.

The PCR reactions were effected with the pairs of primers indicated in Table 1 for the ribosomal Thio16S gene, applying the conditions described in Tables 5 and 6, whereas for the SoxB gene, the pairs of primers indicated in Table 2 were used, applying the conditions described in Tables 5 and 7.

The pairs of primers which, for the ribosomal Thio 16S gene and for the SoxB gene gave the best results with respect to amplification and specificity as diagnostic sequences due to the presence of sulfo-oxidizing bacteria, are indicated below:

Thio16S

| | |
|---|---|
| T9 | GAA AAG GYG GGT YCT AAT A |
| T19 | CAT CTC TGC AAR RTT CCG G |
| T9 | GAA AAG GYG GGT YCT AAT A |
| T21 | CCT GTG TTC CGA TTC CCC GA |
| 360f | GAT CCT GTC GGG NCA YAC SCA YGA |
| 445r | GTT GAA GTT GCC SCG SCG RTA |

Sox B

| | |
|---|---|
| 360f | GAT CCT GTC GGG NCA YAC SCA YGA |
| 531r | GTC GCC GCC TTG YTG RTA RTA |
| 445f | TAT CGG CGS GGC AAY TTC AA |
| 529r | GCC CTG CTG GTA RTA SGG RTC |
| 445f | TAT CGG CGS GGC AAY TTC AA |
| 531R | GTC GCC GCC TTG YTG RTA RTA |

Semiquantitative PCR tests were contemporaneously carried out on environmental samples and on a culture of Thiobacillus consortium consisting of 3 sulfo-oxidizing strains of the wild type obtained from materials taken from sulfur deposits selected in the laboratory (the selection is widely described in Italian patent application MI 03 A 000882 filed by the same Applicant and entitled: Metodo per inibire l'acidificazione biologica dell'acqua in contatto con materiali contenenti zolfo (Method for inhibiting the biological acidification of water in contact with materials containing sulfur) using the pairs of primers SoxB 445f-531r.

These detected not only the presence, but also the variation in the concentration of the Thiobacilli with variations in the quantity of sulfur present in the soil.

The results obtained also show a substantial correlation with those obtained with the MPN method.

The conditions used for the Semiquantitative PCR tests are indicated in Tables 10 and 11.

With the certainty of availing of suitable probes for the detection of sulfo-oxidizing bacteria in the environment, experimentations aimed at setting up a quantitative PCR method were initiated.

The method is based on the fact that the accumulation rate of the gene amplification product is proportional to the quantity of genomic DNA present in the reaction mixture.

It is therefore possible, adopting instruments for the quantitative PCR, to construct a calibration curve using the pairs of primers of the invention mixed with different known concentrations of genomic DNA of sulfo-oxidizing bacteria: to determine the quantity of DNA produced during amplification by means of fluorometry and subsequently calculate by interpolation, the quantity of unknown DNA of the samples to be analyzed.

The amplification is preferably carried out according to the conditions indicated in the SybrGreen™ Kit supplied by Applied Biosystems or according to protocols provided by the producers.

A further advantage of the method described is its easy adaptation to protocols to be used “in situ” such as, for example, the use of portable instruments.

The following examples and figures illustrate the invention without limiting its scope.

### EXAMPLE 1

### Identification of Specific and Preserved Sequences of the Thio16S Gene and SoxB Gene for Selectively Amplifying Sulfo-Oxidizing Bacteria

Various sequences of the Thio16S gene and SoxB gene representative of different species of Thiobacillus bacteria and sulfo-oxidizing bacteria were taken from data banks. Preserved areas were sought, by means of alignment with the use of Clustal X software, in order to outline possible primers for selectively amplifying sulfo-oxidizing bacteria.

The sequences illustrated in FIG. 1 for the Thio16S gene and FIG. 2 for the SoxB gene were thus identified. These sequences were used for the synthesis of degenerated oligonucleotides to be used as amplification primers.

From an examination of homologous areas, different regions of the Thio16S gene were identified with a preserved sequence from which the primers indicated in Table 1 were synthesized.

TABLE 1

| | |
|---|---|
| Primers | Sequence |

| | |
|---|---|
| T1f | CTT GCT AAT TTG CTG ACG AGT |
| T2f | CTT CGG GAT GCT GRC GAG T |
| T3f | AGG TCT TCG GAT GCT GRC GAG T |
| T4f | TAA TAT CGG AAG ATT AAA G |
| T5f | TGA GCC CTR MGG GGG AAA G |
| T6f | TAC GCC CTA CGG GGG AAA G |
| T7f | ATA TGT TAA TAG CGT ATC AA |
| T8f | GAT CCG AAT ACG GTC TGC TA |
| T9f | GAA AAG GYG GGT YCT AAT A |
| T10r | CTT TAA TCT TCC GAT ATT A |
| T11r | CTT TCC CCC KYA GGG CTC A |
| T12r | CTT TCC CCC GTA GGG CGT A |
| T13r | GCT ATT AAC ATA TCA ACC TTC |
| T14r | CGT ATT CGG ATC CGC CTT TTC |
| T15r | TAT TAG RAC CCR CCT TTT CGT |
| T16r | CCA TGG AAT CTT GTA GAG AT |
| T17r | TCC GGA AYY CTG CAG AGA TG |
| T18r | CAT CTC TAC AAG ATT CCA TG |
| T19r | CAT CTC TGC AAR RTT CCG G |
| T20r | CCT GTC TCA TGG TTC CCG AA |
| T21r | CCT GTG TTC CGA TTC CCC GAA |
| T22r | CCT GTC TCT CGG TTC CCG AA |
| T23r | GTG TTC CGG TTC TCT TTC GA |

Table 2 describes the primers of the SoxB gene identified from an examination of the homologous areas of the SoxB except for the primers SoxBf and SoxBr which were suggested by literature (R. Petri and J. Imhoff. Phylogeny and distribution of the SoxB gene among thiosulfate oxidizing bacteria. EMS 197 (2):171-178 (2001)).

TABLE 2

| | |
|---|---|
| soxBf | GAC GGC GGC GAY ACS TGG CAR GG |
| 188f | GAC GAC GCC GAA TGG GAY GAR CC |
| 270f | GGG CAG GCC TTY CCN TAY ATG |
| 360f | GAT CCT GTC GGG NCA YAC SCA YGA |
| 403f | AAA CTG ATC CCG ATC TTY TCG GA |
| 445f | TAT CGG CGS GGC AAY TTC AA |
| 403r | TTC GAR AAG ATC GGG ATC AGY TT |
| 445r | GTT GAA GTT GCC SCG SCG RTA |
| 529r | GCC CTG CTG GTA RTA SGG RTC |
| 531r | GTC GCC GCC TTG YTG RTA RTA |
| soxBr1 | TTC TTC GCC ACY TCY TCY TC |
| soxBr2 | CCA CTC NGG RAA CAT CCA |

### EXAMPLE 2

### Correlation Between Bacterial Growth (MPN), Acidification and Sulfur Presence

In order to verify the correlation between the concentration of Thiobacilli and the quantity of sulfur, the following experiments were effected:

A) with cultures of Thiobacilli consortium and sulfur
B) with soil in the presence of sulfur
A) Thiobacilli consortium and Sulfur Culture

50 ml of medium for Thiobacilli™ containing (g/l): KH₂PO₄ (3.5); (NH₄)₂SO₄ (0.3); MgSO₄7H₂O (0.5); FeSO₄7H₂O (0.018); CaCl₂ (0.25), were charged into a 100 ml flask. The pH value was adjusted to 6 with HCl 0.1N and the TM was sterilized in a vapour autoclave for 20 minutes at 121° C., inoculated with 0.4% (vol/vol) of the bacterial suspension of Thiobacillus consortium obtained as described in the patent cited above.

2.0 g/l of elemental sulfur (in powder at 98%-UNI sieved: 0.05) were added to the TM culture.

The culture thus obtained was incubated under light orbital stirring conditions (100 rpm) for 72 hours at room temperature and used as inoculum.

50 ml of TM with 5% of the above inoculum and different concentrations of sulfur (50, 100, 500, 1000 and 4000 ppm) were charged into 5 100 ml flasks. The cultures were controlled for 20 days by registering the optical density value (at 600 nm) and measuring the pH value. The results are indicated in Table 3.

TABLE 3

| | Time (days) | | | | | | | |
|---|---|---|---|---|---|---|---|---|
| Sulfur | 0 | | 4 | | 10 | | 20 | |
| ppm | O.D. | pH | O.D. | pH | O.D. | pH | O.D. | pH |
| 50 | 0.032 | 6.0 | 0.042 | 6.0 | 0.08 | 5.8 | 0.08 | 5.8 |
| 100 | 0.032 | 6.0 | 0.040 | 6.0 | 0.076 | 6.0 | 0.081 | 5.8 |
| 500 | 0.035 | 6.0 | 0.072 | 6.0 | 0.182 | 2.5 | 0.458 | 1.8 |
| 1000 | 0.038 | 6.0 | 0.078 | 6.0 | 0.190 | 2.5 | 0.481 | 1.8 |
| 4000 | 0.038 | 6.0 | 0.082 | 6.0 | 0.192 | 2.5 | 0.507 | 1.8 |

From Table 3 it can be seen that sulfur at a value of 500 ppm up to 4000 ppm is capable of causing an increase in the growth of Thiobacilli and the acidification of the culture medium, below the value of 500 ppm there is no significant variation either in the O.D. or pH.

### B) Soil in the Presence of Sulfur.

5 small containers (10×15 cm) were prepared, each containing 250 g of soil (sieved at 1.4 mm taken from the EniTecnologie centre) and 50 g of soil (sieved at 1.4 mm taken from the EniTecnologie centre) polluted with varying quantities of sulfur (10, 50, 100, 500 and 4000 ppm). 20 ml of rainwater were then added (with a sprayer) maintaining this volume constant.

Table 4 shows the results of the growth of Thiobacilli, evaluated after 20 days at room temperature with the MPN technique.

TABLE 4

| Sulfur (ppm) | MPN/g |
|---|---|
| 1.67 | <3 |
| 8.33 | <3 |
| 16.67 | <3 |
| 83.33 | >10⁵ |
| 666.67 | >10⁵ |

The results show that the MPN technique allows the presence of Thiobacilli to be detected starting from a sulfur concentration of about 83 ppm.

The MPN technique can therefore be used for detecting the presence of traces of sulfur, for monitoring the environment using Thiobacilli as “biomarkers”.

The MPN technique was used (E. Cochran. Estimation of bacterial densities by means of the “most probable number”. Biometrics 6:105-116 (1950)) using TM at pH 6. In practice, the samples of soil were diluted in Ringer solution (5 g in 45 ml) and a scaled series was prepared of decimal dilutions (1 ml of sample diluted in 9 ml of Ringer solution).

For each dilution 1 ml of soil ringer dilution was transferred to 5 ml of TM medium with 0.02 g (0.4%) of elemental sulfur (98% sulfur in powder form, sieved UNI 0.05) and 3 15 ml tubes were prepared for each dilution.

Two methods suggested by literature were then applied:

I—in which the positivity of the sulfo-oxidizing bacteria is based on the wettability of the sulfur and the turbidity of the culture medium (C. Knickerbocker. The role of blebbing in overcoming the hydrophobic barrier during biooxidation of elemental sulfur by Thiobacillus thiooxidans. Chemical Geology 169: 425-433 (2000)).
II—the positivity of the sample was given by the toning of the pH and consequent colour change of the culture medium due to the addition of a pH indicator 15 ml/l (purple bromocresol at 15%) to the TM (M. Hines. Sulfphur cycling, Manual of Environmental Microbiology (1998)).

The second method is preferable as it is more rapid and easier to interpret with respect to the first.

### EXAMPLE 3

### Determination of Sulfo-Oxidizing Bacteria in the Soil and Culture of Thiobacillus consortium with Probes Based on the Thio 16S and SoxB Gene

The sample to be analyzed consisted of DNA coming from environmental samples (soil) and from bacterial cultures of Thiobacillus consortium.

Various methods were used for the extraction of the bacterial DNA:

from a culture of Thiobacillus (grown for 72 hours); the DNA is extracted with the CTAB/NaCl method and re-suspended in 50 μl of TE (Tris-Edta).

from environmental samples; this method starts from 500 mg of soil and the Fast DNA Kit (BIO 101) is used, resuspending the DNA in a finale volume of 70 μl of DES (DNA Elution Solution).

The PCR reaction was carried out with various combinations of pairs of primers for Thio 16S on soil and on a Thiobacillus consortium culture following the conditions described in Table 5, and various combinations of pairs of primers for Sox B on Thiobacillus consortium following the conditions described in Table 5 and 7.

TABLE 5

| Reagent | Volume | | Final concentration |
|---|---|---|---|
| PCR buffer 10x | 2.5 | μl | 2.5 mM |
| Primers forward | 1 | μl | 4 pmoles |
| Primers reverse | 1 | μl | 4 pmoles |
| DNTPS (10 mM each) | 0.5 | μl | 0.2 mM |
| DNA | 1 | μl | 1-5 ng/μl |
| Taq DNA pol | 0.5 | μl | 0.1 U |
| Water | up to 2.5 | μl | — |

TABLE 6

| Nr. cycles | T ° C. | Time |
|---|---|---|
| 30 | 94 | 1′ |
| | 55 | 50″ |
| | 72 | 1′ |

TABLE 7

| Nr. cycles | T ° C. | Time |
|---|---|---|
| 30 | 94 | 1′ |
| | 57 | 50″ |
| | 72 | 1′ |

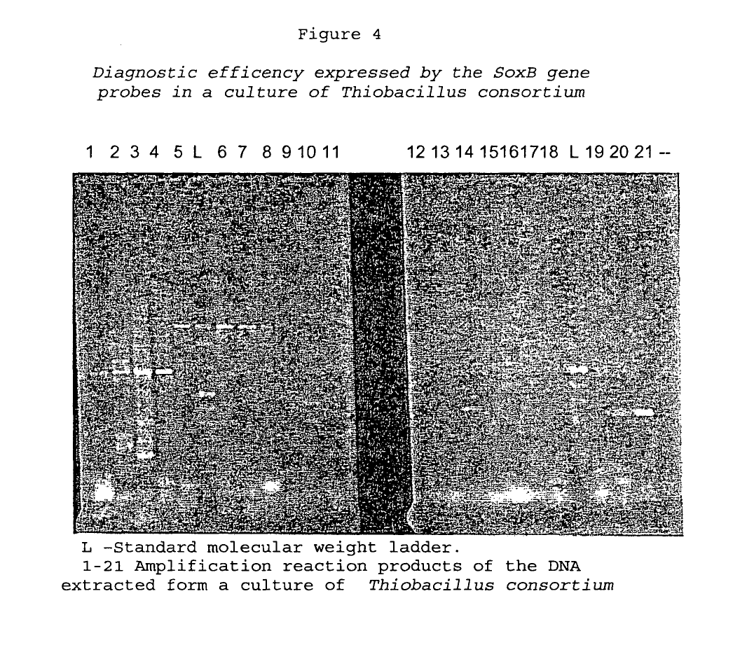

Following the above procedures, the results obtained, after analysis on agarose gel, are indicated in Table 8 relating to the Thio 16S gene and in Table 9 and in FIG. 4 relating to the SoxB gene.

TABLE 8

| Primer F | | | Expected |
|---|---|---|---|
| Primer R | Th consortium | Soil | results |
| T1-T10 | +/− | +/− | 122 bp |
| T4-T13 | +/− | +/− | 308 bp |
| T4-T20 | +/− | +/− | 879 bp |
| T7-T18 | +/− | +/− | 558 bp |
| T7-T20 | +/− | +/− | 591 bp |
| T4-T18 | +/− | +/− | 846 bp |
| T1-T13 | +/− | +/− | 411 bp |
| T1-T18 | +/− | +/− | 949 bp |
| T9-T19 | OK | OK | 558 bp |
| T9-T21 | OK | OK | 591 bp |
| T1-T20 | +/− | +/− | 982 bp |
| T3-T11 | +/− | +/− | 122 bp |
| T3-T15 | +/− | +/− | 411 bp |
| T3-T19 | +/− | +/− | 949 bp |
| T3-T21 | +/− | +/− | 982 bp |
| T5-T15 | +/− | +/− | 308 bp |
| T5-T19 | +/− | +/− | 846 bp |
| T5-T21 | +/− | +/− | 879 bp |

OK = obtaining the expected amplification product as a single band or as a predominant band
+/− = obtaining aspecific bands

TABLE 9

| Primer | Primer | | Lane in |
|---|---|---|---|
| Forward | Reverse | Expected results | FIG. 4 |
| soxB | 403 | 633 bp | 1 |
| soxB | 445 | 759 bp | 2 |
| soxB | 529 | 1011 bp | 3 |
| soxB | 531 | 1020 bp | 4 |
| 188 | 403 | 438 bp | 5 |
| 188 | 445 | 554 bp | 6 |
| 188 | 529 | 816 bp | 7 |
| 188 | 531 | 825 bp | 8 |
| 270 | 403 | 357 bp | 9 |
| 270 | 445 | 483 bp | 10 |
| 270 | 529 | 735 bp | 11 |
| 270 | 531 | 744 bp | 12 |
| 360 | 403 | 135 bp | 13 |
| 360 | 445 | 243 bp | 14 |
| 360 | 529 | 495 bp | 15 |
| 360 | 531 | 504 bp | 16 |
| 403 | 445 | 150 bp | 17 |
| 403 | 529 | 387 bp | 18 |
| 403 | 531 | 396 bp | 19 |
| 445 | 529 | 249 bp | 20 |
| 445 | 531 | 255 bp | 21 |

Two pairs of primers T9-T21 and T9-T19 were then identified, capable of giving positive amplification and specificity results for use as diagnostic sequences for the presence and quantification of Thiobacilli.

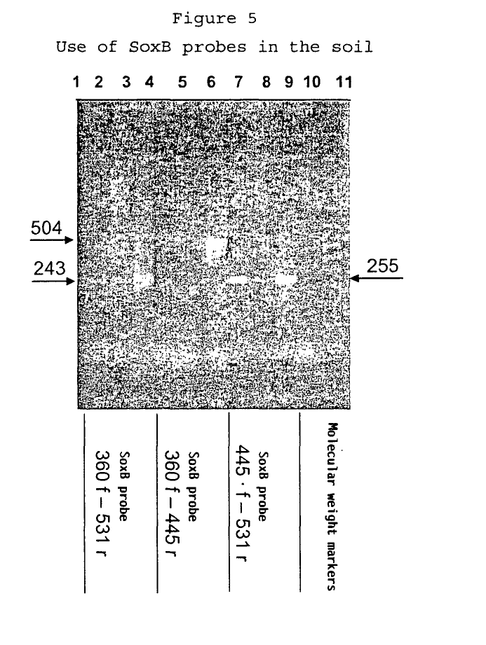

Analysis of the pattern of the amplification products for the SoxB gene in a culture of Thiobacillus consortium, shown in FIG. 4, indicates the pairs of primers used in the reactions 14, 16, 20 and 21 as being the most specific and efficient. The primers 360f, 445f, 445r, 529r, 531r can therefore be used for the monitoring of S-oxidizing bacteria in the soil (FIG. 5).

It should also be pointed out that the response obtained with the SoxB primers, used in literature for a philogenetic survey of aquatic microbial species (Chlorobium and Paracoccus) involved in the sulfur cycle in a river estuary, is absolutely inadequate for the use of these primers in quantitative PCR reactions as the presence of aspecific bands (see for example FIG. 4) and primer-dimers has been observed. It can therefore be asserted that the probes identified, object of the present invention, are more efficient for detecting sulfo-oxidizing bacteria in the soil and in environmental matrixes in general.

### EXAMPLE 4

### Semiquantitative PCR Tests on Environmental Samples and on a Culture of Thiobacillus consortium

In order to evaluate the possibility of use of the SoxB 445f-531r gene probe to effect Semi-quantitative analyses on the presence of Thiobacilli in the soil, a comparative test was prepared of the amplification products obtained (at different dilutions) from the DNA extracted from:

soil exposed to sulfur contamination (Sample H taken from pertinent areas of the EniTecnologie laboratories near a sulfur-emitting source

common soil (Sample D)

a culture of reference bacteria (Thiobacillus consortium).

The semi-quantitative PCR reaction was carried out following the conditions described in Tables 10 and 11.

TABLE 10

| Reagent | Volume | | Final concentration |
|---|---|---|---|
| PCR buffer 10x | 2.5 | μl | 2.5 mM |
| Primers forward | 1 | μl | 4 pmoles |
| Primers reverse | 1 | μl | 4 pmoles |
| DNTPs (10 mM each) | 0.5 | μl | 0.2 mM |
| DNA | 1 | μl | 1-5 ng/μl |
| Taq DNA pol | 0.5 | μl | 0.1 U |
| Water | up to 25 | μl | — |

TABLE 11

| Nr. of cycles | T C. | Time |
|---|---|---|
| 18 | 94 | 1′ |
| | 55 | 50″ |
| | 72 | 1′ |
| 21 | 94 | 1′ |
| | 55 | 50″ |
| | 72 | 1′ |
| 25 | 94 | 1′ |
| | 55 | 50″ |
| | 72 | 1′ |
| 28 | 94 | 1′ |
| | 55 | 50″ |
| | 72 | 1′ |
| 30 | 94 | 1′ |
| | 55 | 50″ |
| | 72 | 1′ |

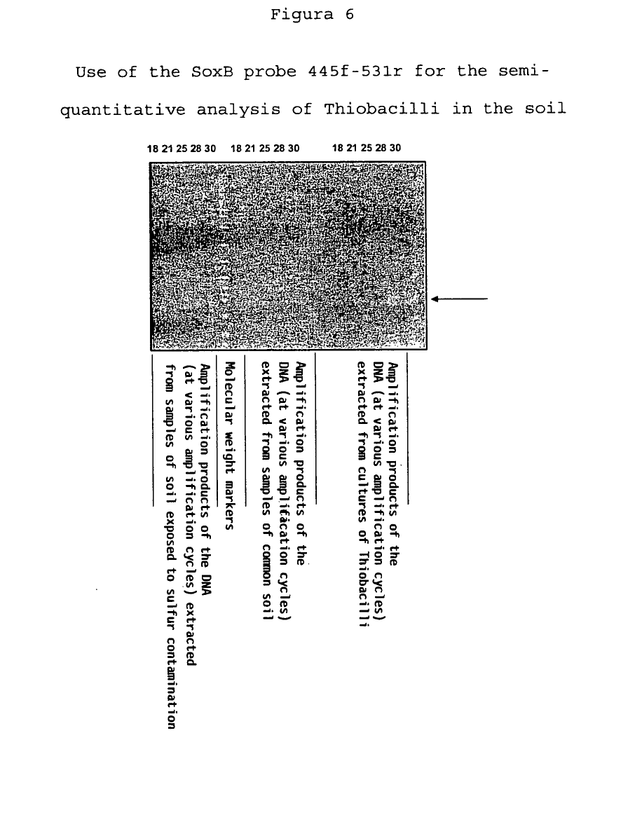

The results obtained after analysis on agarose gel are indicated in FIG. 6.

These tests indicate a relatively higher concentration of sulfo-oxidizing bacteria in a point definitely contaminated by Sₒ (Sample H) with respect to other types of soil (sample D) and are convalidated by the results obtained with the MPN which are indicated in Table 12.

TABLE 12

| Sample | MPN/g |
|---|---|
| H | 10⁷-10⁹ |
| D | 10⁵-10⁶ |

Both of the methods (MPN and PCR) are able to demonstrate not only the presence of Thiobacilli in the soil, but also a variation in their concentration.

The primers 445f and 531r were also successfully used in quantitative PCR experiments for effecting the gene dosage of SoxB in environmental samples.

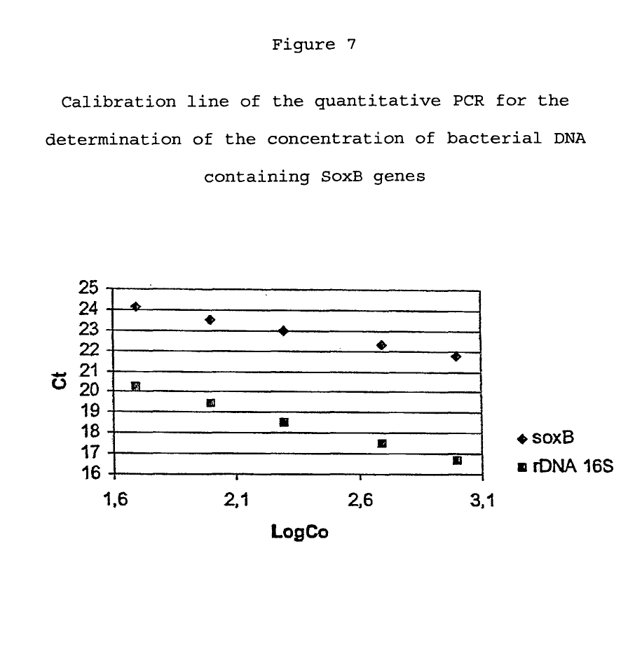

For this purpose, a calibration curve (standard) was constructed, using the pair of specific primers 445f and 531r for the SoxB gene and starting from the DNA extracted from environmental samples containing sulfo-oxidizing bacteria, diluted at various known concentrations (see rhomboidal symbols indicated in FIG. 7).

This allows the presence and quantity of DNA containing SoxB genes present in various types of environmental samples (water, soil, etc.) to be traced, through interpolation.

The signal obtained with primers for eubacteria (see the square symbols indicated in FIG. 7) based on 16S rDNA sequences reveals all the bacteria present in the sample.

Reactions without DNA or reactions on DNA extracted from samples in which S-oxidative bacteria are absent do not give a positive signal.

1. A method for the identification of sulfo-oxidizing bacteria comprising extraction of DNA from environmental samples and subsequent identification of at least one fragment of the ribosomal Thio 16S gene and/or the SoxB gene present in the bacteria.

2. The method according to claim 1, wherein the identification of the fragment of the Thio 16S gene is conducted by gene amplification in the presence of pairs of oligonucleotides complementary to the Thio 16S gene selected from the following pairs of sequences:

| | | |
|---|---|---|
| T 9 | GAA AAG GYG GGT YCT AAT A | (SEQ ID NO: 1) |
| T19 | CAT CTC TGC AAR RTT CCG G | (SEQ ID NO: 2) |
| T 9 | GAA AAG GYG GGT YCT AAT A | (SEQ ID NO: 1) |
| T 21 | CCT GTG TTC CGA TTC CCC GA, | (SEQ ID NO: 3) |

whereas the identification of the SoxB gene is conducted by gene amplification in the presence of pairs of oligonucleotides complementary to the SoxB gene in which the primer forward is selected from the following sequences:

| | | |
|---|---|---|
| 360f | GAT CCT GTC GGG NCA YAC SCA | (SEQ ID NO: 4) |
| | YGA | |
| 445f | TAT CGG CGS GGC AAY TTC AA | (SEQ ID NO: 5) |

and the primer reverse is selected from the following sequences:

| | | |
|---|---|---|
| 445r | GTT GAA GTT GCC SCG SCG RTA | (SEQ ID NO: 6) |
| 531r | GTC GCC GCC TTG YTG RTA RTA | (SEQ ID NO: 7) |
| 529r | GCC CTG CTG GTA RTA SGG RTC. | (SEQ ID NO: 8) |

3. The method according to claim 2, wherein the gene amplification of the SoxB gene is conducted in the presence of pairs of oligonucleotides selected from the following pairs of sequences:

| | | |
|---|---|---|
| 360f | GAT CCT GTC GGG NCA YAC SCA YGA | (SEQ ID NO: 4) |
| 445r | GTT GAA GTT GCC SCG SCG RTA | (SEQ ID NO: 6) |
| 360f | GAT CCT GTC GGG NCA YAC SCA YGA | (SEQ ID NO: 4) |
| 531r | GTC GCC GCC TTG YTG RTA RTA | (SEQ ID NO: 7) |
| 445f | TAT CGG CGS GGC AAY TTC AA | (SEQ ID NO: 5) |
| 529r | GCC CTG CTG GTA RTA SGG RTC | (SEQ ID NO: 8) |
| 445f | TAT CGG CGS GGC AAY TTC AA | (SEQ ID NO: 5) |
| 531r | GTC GCC GCC TTG YTG RTA RTA. | (SEQ ID NO: 7) |

4. The method for the identification of sulfo-oxidizing bacteria according to claim 1, comprising:
extracting the DNA from the samples;
putting the extracted DNA in contact with a pair of primers, under conditions which allow specific annealing and amplification of the fragment of the Thio 16S or SoxB gene;
amplifying the fragment of the Thio16S or SoxB gene; and
analyzing the gene amplification product by a real-time PCR or gel-electrophoresis, wherein the pair of primers complementary to the Thio 16S gene is selected from the following pairs of sequences:

| | | |
|---|---|---|
| T9 | GAA AAG GYG GGT YCT AAT A | (SEQ ID NO: 1) |
| T19 | CAT CTC TGC AAR RTT CCG G | (SEQ ID NO: 2) |
| T9 | GAA AAG GYG GGT YCT AAT A | (SEQ ID NO: 1) |
| T21 | CCT GTG TTC CGA TTC CCC GA, | (SEQ ID NO: 3) |

and the pair of primers complementary to the SoxB gene in which the primer forward is selected from the following sequences:

| | | |
|---|---|---|
| 360f | GAT CCT GTC GGG NCA YAC SCA YGA | (SEQ ID NO: 4) |
| 445f | TAT CGG CGS GGC AAY TTC AA | (SEQ ID NO: 5) |

and the primer reverse is selected from the following sequences:

| | | |
|---|---|---|
| 445r | GTT GAA GTT GCC SCG SCG RTA | (SEQ ID NO: 6) |
| 531r | GTC GCC GCC TTG YTG RTA RTA | (SEQ ID NO: 7) |
| 529r | GCC CTG CTG GTA RTA SGG RTC. | (SEQ ID NO: 8) |

5. The method for the identification of sulfo-oxidizing bacteria according to claim 4, wherein the annealing temperatures in the phase preceding the specific gene amplification, range from −5 to −1° C. with respect to the lowest melting temperature Tm among those of the pair of probes used and the polymerization time is a minute of reaction per kilobase of DNA polymerized.

6. A method for the quantitative determination of sulfo-oxidizing bacteria comprising:
performing a gene amplification according to the method of claim 4 in the presence of different quantities of genomic DNA of sulfo-oxidizing bacteria;
determining quantity of the gene amplification product;
constructing a calibration curve; and
determining quantity of the genomic DNA in samples to be analyzed by means of interpolation.

7. The method according to claim 4, wherein the gene amplification is carried out in the presence of the pair of oligonucleotides complementary to the SoxB gene having the following pair of sequences:

| | | |
|---|---|---|
| 445f | TAT CGG CGS GGC AAY TTC AA | (SEQ ID NO: 5) |
| 531r | GTC GCC GCC TTG YTG RTA RTA. | (SEQ ID NO: 7) |

8. A method for determining dispersion of sulfur in an environment comprising identifying sulfo-oxidizing bacteria and determining quantity of the bacteria.

9. The method according to claim 8, wherein the quantitative determination of said sulfo-oxidizing bacteria is conducted by a method comprising:
performing a gene amplification in the presence of different quantities of genomic DNA of the sulfo-oxidizing bacteria;
determining quantity of the gene amplification product;
constructing a calibration curve; and
determining quantity of the genomic DNA in samples to be analyzed by means of interpolation.

10. A diagnostic kit for identifying the presence of sulfo-oxidizing bacteria in environmental samples based on the identification of the ThioS or SoxB genes.

11. A pair of oligonucleotides complementary to the Thio16S gene of sulfo-oxidizing bacteria selected from the following pairs of sequences:

| | | |
|---|---|---|
| T9 | GAA AAG GYG GGT YCT AAT A | (SEQ ID NO: 1) |
| T19 | CAT CTC TGC AAR RTT CCG G | (SEQ ID NO: 2) |
| T9 | GAA AAG GYG GGT YCT AAT A | (SEQ ID NO: 1) |
| T21 | CCT GTG TTC CGA TTC CCC GA. | (SEQ ID NO: 3) |

12. A pair of oligonucleotides complementary to the SoxB gene of sulfo-oxidizing bacteria selected from the following pairs of sequences:

| | | |
|---|---|---|
| 360f | GAT CCT GTC GGG NCA YAC SCA YGA | (SEQ ID NO: 4) |
| 445r | GTT GAA GTT GCC SCG SCG RTA | (SEQ ID NO: 6) |
| 360f | GAT CCT GTC GGG NCA YAC SCA YGA | (SEQ ID NO: 4) |
| 531r | GTC GCC GCC TTG YTG RTA RTA | (SEQ ID NO: 7) |
| 445f | TAT CGG CGS GGC AAY TTC AA | (SEQ ID NO: 5) |
| 529r | GCC CTG CTG GTA RTA SGG RTC | (SEQ ID NO: 8) |
| 445f | TAT CGG CGS GGC AAY TTC AA | (SEQ ID NO: 5) |
| 531r | GTC GCC GCC TTG YTG RTA RTA. | (SEQ ID NO: 7) |

13. An oligonucleotide complementary to the ThioS gene of sulfo-oxidizing bacteria, selected from one of the following sequences:

| | | |
|---|---|---|
| T9 | GAA AAG GYG GGT YCT AAT A | (SEQ ID NO: 1) |
| T19 | CAT CTC TGC AAR RTT CCG G | (SEQ ID NO: 2) |
| T9 | GAA AAG GYG GGT YCT AAT A | (SEQ ID NO: 1) |
| T21 | CCT GTG TTC CGA TTC CCC GA. | (SEQ ID NO: 3) |

14. An oligonucleotide complementary to the SoxB gene of sulfo-oxidizing bacteria, selected from one of the following sequences:

| | | |
|---|---|---|
| 360f | GAT CCT GTC GGG NCA YAC SCA YGA | (SEQ ID NO: 4) |
| 445r | GTT GAA GTT GCC SCG SCG RTA | (SEQ ID NO: 6) |
| 360f | GAT CCT GTC GGG NCA YAC SCA YGA | (SEQ ID NO: 4) |
| 531r | GTC GCC GCC TTG YTG RTA RTA | (SEQ ID NO: 7) |
| 445f | TAT CGG CGS GGC AAY TTC AA | (SEQ ID NO: 5) |
| 529r | GCC CTG CTG GTA RTA SGG RTC | (SEQ ID NO: 8) |
| 445f | TAT CGG CGS GGC AAY TTC AA | (SEQ ID NO: 5) |
| 531r | GTC GCC GCC TTG YTG RTA RTA. | (SEQ ID NO: 7) |

15. A method for identifying sulfo-oxidizing bacteria comprising hybridizing a labeled probe with genomic DNA of a sample to be analyzed, wherein the probe consists of at least one of the following sequences:

| | | |
|---|---|---|
| T9 | GAA AAG GYG GGT YCT AAT A | (SEQ ID NO: 1) |
| T19 | CAT CTC TGC AAR RTT CCG G | (SEQ ID NO: 2) |
| T9 | GAA AAG GYG GGT YCT AAT A | (SEQ ID NO: 1) |
| T21 | CCT GTG TTC CGA TTC CCC GA. | (SEQ ID NO: 3) |
| 360f | GAT CCT GTC GGG NCA YAC SCA YGA | (SEQ ID NO: 4) |
| 445r | GTT GAA GTT GCC SCG SCG RTA | (SEQ ID NO: 6) |
| 360f | GAT CCT GTC GGG NCA YAC SCA YGA | (SEQ ID NO: 4) |
| 531r | GTC GCC GCC TTG YTG RTA RTA | (SEQ ID NO: 7) |
| 445f | TAT CGG CGS GGC AAY TTC AA | (SEQ ID NO: 5) |
| 529r | GCC CTG CTG GTA RTA SGG RTC | (SEQ ID NO: 8) |
| 445f | TAT CGG CGS GGC AAY TTC AA | (SEQ ID NO: 5) |
| 531r | GTC GCC GCC TTG YTG RTA RTA. | (SEQ ID NO: 7) |

16. The method according to claim 15, wherein the genomic DNA consists of the gene amplification product obtained by amplification of the Thio16S gene in the presence of pairs of oligonucleotides complementary to the Thio 16S gene selected from the following pairs of sequences:

| | | |
|---|---|---|
| T9 | GAA AAG GYG GGT YCT AAT A | (SEQ ID NO: 1) |
| T19 | CAT CTC TGC AAR RTT CCG G | (SEQ ID NO: 2) |
| T9 | GAA AAG GYG GGT YCT AAT A | (SEQ ID NO: 1) |
| T21 | CCT GTG TTC CGA TTC CCC GA, | (SEQ ID NO: 3) |

and/or the SoxB gene in the presence of pairs of oligonucleotides complementary to the SoxB gene in which the primer forward is selected from the following sequences:

| | | |
|---|---|---|
| 360f | GAT CCT GTC GGG NCA YAC SCA | (SEQ ID NO: 4) |
| | YGA | |
| 445f | TAT CGG CGS GGC AAY TTC AA | (SEQ ID NO: 5) |

and the primer reverse is selected from the following sequences:

| | | |
|---|---|---|
| 445r | GTT GAA GTT GCC SCG SCG RTA | (SEQ ID NO: 6) |
| 531r | GTC GCC GCC TTG YTG RTA RTA | (SEQ ID NO: 7) |
| 529r | GCC CTG CTG GTA RTA SGG RTC. | (SEQ ID NO: 8) |


SEQUENCE LISTING (36 sequences)

SEQ ID NO: 1 - DNA, 19 bp, Artificial Sequence
  Description of Artificial Sequence Synthetic primer
gaaaaggygggtyctaata

SEQ ID NO: 2 - DNA, 19 bp, Artificial Sequence
  Description of Artificial Sequence Synthetic primer
catctctgcaarrttccgg

SEQ ID NO: 3 - DNA, 20 bp, Artificial Sequence
  Description of Artificial Sequence Synthetic primer
cctgtgttccgattccccga

SEQ ID NO: 4 - DNA, 24 bp, Artificial Sequence
  Description of Artificial Sequence Synthetic primer
gatcctgtcgggncayacscayga

SEQ ID NO: 5 - DNA, 20 bp, Artificial Sequence
  Description of Artificial Sequence Synthetic primer
tatcggcgsggcaayttcaa

SEQ ID NO: 6 - DNA, 21 bp, Artificial Sequence
  Description of Artificial Sequence Synthetic primer
gttgaagttgccscgscgrta

SEQ ID NO: 7 - DNA, 21 bp, Artificial Sequence
  Description of Artificial Sequence Synthetic primer
gtcgccgccttgytgrtarta

SEQ ID NO: 8 - DNA, 21 bp, Artificial Sequence
  Description of Artificial Sequence Synthetic primer
gccctgctggtartasggrtc

SEQ ID NO: 9 - DNA, 21 bp, Artificial Sequence
  Description of Artificial Sequence Synthetic primer
cttgctaatttgctgacgagt

SEQ ID NO: 10 - DNA, 19 bp, Artificial Sequence
  Description of Artificial Sequence Synthetic primer
cttcgggatgctgrcgagt

SEQ ID NO: 11 - DNA, 22 bp, Artificial Sequence
  Description of Artificial Sequence Synthetic primer
aggtcttcggatgctgrcgagt

SEQ ID NO: 12 - DNA, 19 bp, Artificial Sequence
  Description of Artificial Sequence Synthetic primer
taatatcggaagattaaag

SEQ ID NO: 13 - DNA, 19 bp, Artificial Sequence
  Description of Artificial Sequence Synthetic primer
tgagccctrmgggggaaag

SEQ ID NO: 14 - DNA, 19 bp, Artificial Sequence
  Description of Artificial Sequence Synthetic primer
tacgccctacgggggaaag

SEQ ID NO: 15 - DNA, 20 bp, Artificial Sequence
  Description of Artificial Sequence Synthetic primer
atatgttaatagcgtatcaa

SEQ ID NO: 16 - DNA, 20 bp, Artificial Sequence
  Description of Artificial Sequence Synthetic primer
gatccgaatacggtctgcta

SEQ ID NO: 17 - DNA, 19 bp, Artificial Sequence
  Description of Artificial Sequence Synthetic primer
ctttaatcttccgatatta

SEQ ID NO: 18 - DNA, 19 bp, Artificial Sequence
  Description of Artificial Sequence Synthetic primer
ctttccccckyagggctca

SEQ ID NO: 19 - DNA, 19 bp, Artificial Sequence
  Description of Artificial Sequence Synthetic primer
ctttcccccgtagggcgta

SEQ ID NO: 20 - DNA, 21 bp, Artificial Sequence
  Description of Artificial Sequence Synthetic primer
gctattaacatatcaaccttc

SEQ ID NO: 21 - DNA, 21 bp, Artificial Sequence
  Description of Artificial Sequence Synthetic primer
cgtattcggatccgccttttc

SEQ ID NO: 22 - DNA, 21 bp, Artificial Sequence
  Description of Artificial Sequence Synthetic primer
tattagracccrccttttcgt

SEQ ID NO: 23 - DNA, 20 bp, Artificial Sequence
  Description of Artificial Sequence Synthetic primer
ccatggaatcttgtagagat

SEQ ID NO: 24 - DNA, 20 bp, Artificial Sequence
  Description of Artificial Sequence Synthetic primer
tccggaayyctgcagagatg

SEQ ID NO: 25 - DNA, 20 bp, Artificial Sequence
  Description of Artificial Sequence Synthetic primer
catctctacaagattccatg

SEQ ID NO: 26 - DNA, 20 bp, Artificial Sequence
  Description of Artificial Sequence Synthetic primer
cctgtctcatggttcccgaa

SEQ ID NO: 27 - DNA, 21 bp, Artificial Sequence
  Description of Artificial Sequence Synthetic primer
cctgtgttccgattccccgaa

SEQ ID NO: 28 - DNA, 20 bp, Artificial Sequence
  Description of Artificial Sequence Synthetic primer
cctgtctctcggttcccgaa

SEQ ID NO: 29 - DNA, 20 bp, Artificial Sequence
  Description of Artificial Sequence Synthetic primer
gtgttccggttctctttcga

SEQ ID NO: 30 - DNA, 23 bp, Artificial Sequence
  Description of Artificial Sequence Synthetic primer
gacggcggcgayacstggcargg

SEQ ID NO: 31 - DNA, 23 bp, Artificial Sequence
  Description of Artificial Sequence Synthetic primer
gacgacgccgaatgggaygarcc

SEQ ID NO: 32 - DNA, 21 bp, Artificial Sequence
  Description of Artificial Sequence Synthetic primer
gggcaggccttyccntayatg

SEQ ID NO: 33 - DNA, 23 bp, Artificial Sequence
  Description of Artificial Sequence Synthetic primer
aaactgatcccgatcttytcgga

SEQ ID NO: 34 - DNA, 23 bp, Artificial Sequence
  Description of Artificial Sequence Synthetic primer
ttcgaraagatcgggatcagytt

SEQ ID NO: 35 - DNA, 20 bp, Artificial Sequence
  Description of Artificial Sequence Synthetic primer
ttcttcgccacytcytcytc

SEQ ID NO: 36 - DNA, 18 bp, Artificial Sequence
  Description of Artificial Sequence Synthetic primer
ccactcnggraacatcca